(19) United States
(12) Patent Application Publication
(10) Pub. No.: US 2025/0367391 A1
(43) Pub. Date: Dec. 4, 2025

(54) ATHLETIC MOUTHGUARD HAVING AN INTEGRATED INHALER RESPIRATOR

(71) Applicant: Christopher Rotondo, Miami Beach, FL (US)

(72) Inventor: Christopher Rotondo, Miami Beach, FL (US)

(21) Appl. No.: 18/439,347

(22) Filed: Feb. 12, 2024

(51) Int. Cl. A61M 15/00

(52) U.S. Cl. CPC A61M 15/0021

(57) ABSTRACT

A mouthguard including an inhaler respirator to deliver a medication is disclosed. The device includes a mouthguard and an inhaler respirator including one or more clips to removably coupled to the mouthguard. A propellant is stored within the inhaler respirator to expel a medication. The mouthguard allows for the on-demand administration of the medication to provide convenience while engaging in athletic activities.

### TECHNICAL FIELD

The embodiments disclosed herein generally relate to mouthguards and more particularly to mouthguards having specialized attachments and features.

### BACKGROUND

Mouthguards are commonly used during athletic or potentially dangerous activities to protect the teeth, jaw, and gums of the wearers mouth. Traditional mouthguards are simple devices which are constructed of a semi-soft material which reduces the likelihood of injury to the mouth resulting from violent impacts to the users face and head, or accidental biting.

Inhalers have long been used to administer medication into the lungs of the patient. This allows medicines to be delivered to and absorbed in the lungs, which provides the ability for targeted medical treatment to this specific region of the body, as well as a reduction in the side effects of oral medications. Athletes who suffer from illness and/or disease which require or benefit from inhaled medicine are often inconvenienced by the utilization of inhalers, which are not always stored on their person and may be inaccessible due to location and equipment restraints.

### SUMMARY OF THE INVENTION

This summary is provided to introduce a variety of concepts in a simplified form that is further disclosed in the detailed description of the embodiments. This summary is not intended for determining the scope of the claimed subject matter.

A mouthguard including an inhaler respirator to deliver a medication is disclosed. The device includes a mouthguard and an inhaler respirator including one or more clips to removably coupled to the mouthguard. A propellant is stored within the inhaler respirator to expel a medication. The mouthguard allows for the on-demand administration of the medication to provide convenience while engaging in athletic activities. In such, the user does not need to remove equipment to access their mouth to administer the medication. Further, the user does not need to find their inhaler which may not necessarily be carried on their person during the athletic activity.

The device may be used to prevent and treat negative reactions and symptoms of asthma induced attacks to athletes by emitting medication from the inhaler respirator. The medication may not necessarily be a medication for treating asthma but may be another medication or supplement to treat other diseases and conditions. The medication may also be used to enhance athletic performance or provide other benefits.

In one aspect, the mouthguard is comprised of an inner wall, an outer wall, and a bottom to form a channel.

In one aspect, the inner wall forms a friction fit with the one or more clips to retain the inhaler respirator in position.

In one aspect, the inhaler respirator includes a button to be selectively pressed to expel the medication.

In one aspect, the medication is expelled through one or more openings positioned on a rear side of the inhaler respirator.

In one aspect, the one or more clips protrude perpendicular to the top surface of the inhaler respirator. In one aspect, the propellant is carbon dioxide or other suitable propellant.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates an exploded view of the mouthguard and inhaler respirator, wherein the inhaler respirator is separated from the mouthguard, according to some embodiments;

FIG. 2 illustrates a perspective view of the top of the mouthguard and inhaler respirator, wherein the inhaler respirator is coupled to the mouthguard, according to some embodiments;

FIG. 3 illustrates a perspective view of the mouthguard and inhaler respirator expelling a medication, according to some embodiments; and

FIG. 4 illustrates a perspective view of the bottom of the mouthguard and inhaler respirator, wherein the inhaler respirator is coupled to the mouthguard, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided herein relate to an athletic mouthguard having an integrated inhaler respirator (which hereinafter may be referred to as the “device”) to provide the on-demand administration of an inhalant. The device includes a mouthguard configured in a similar manner to a traditional mouthguard commonly worn by athletes or other individuals wanting to protect their mouth (including the teeth, tongue, gums, jaw, and other anatomical structures of the mouth).

As used herein, the term “mouth” can refer to anatomical structure within or around the mouth including the teeth, tongue, gums, jaw, lips, etc.

As used herein, the term “device” can be used to describe both the mouthguard and inhaler respirator when they are coupled together.

The device may prevent and treat negative reactions and symptoms of asthma induced attacks to athletes by emitting medication from the inhaler respirator. One skilled in the arts will readily understand that the medication may not necessarily be a medication for treating asthma but may be another medication or supplement to treat other diseases and conditions. The medication may also be used to enhance athletic performance (in the embodiments wherein the medication is a performance enhancing supplement).

The device helps athletes or other users feel at-ease, knowing that they are prepared for an asthma attack or the symptoms thereof as the device provides the on-demand administration of the medication without the need to remove equipment (e.g., a helmet), or go find their medication which may not be carried on their person. The device provides instant access to medicated, FDA regulated, and NCAA regulated inhalant medicine to treat symptoms, open up lungs and continue performing at their peak performance.

During use, the user attaches the inhaler respirator to the mouthguard. The mouthguard fits within the mouth as any traditional mouthguard would normally fit. To emit the medicine from the inhaler respirator, a button is provided on the inhaler respirator to expel the medicine therefrom. The button may be accessed by opening the mouth and pressing the button with the user's thumb or other finger. In some embodiments, the user may hold the button for a period of time (such as three seconds) to release a premeasured dose of the medication. In some instances, the user may take a breath to draw the medication into the lungs.

In some embodiments, the inhaler respirator includes a medication cartridge to provide storage for the medication. This cartridge may contain one or more effective doses of the medication. In one example, the cartridge may contain between 2-5 effective doses of the medication. The medication may be expelled using a carbon dioxide cartridge, or other means of forcibly delivering the medication known in the arts.

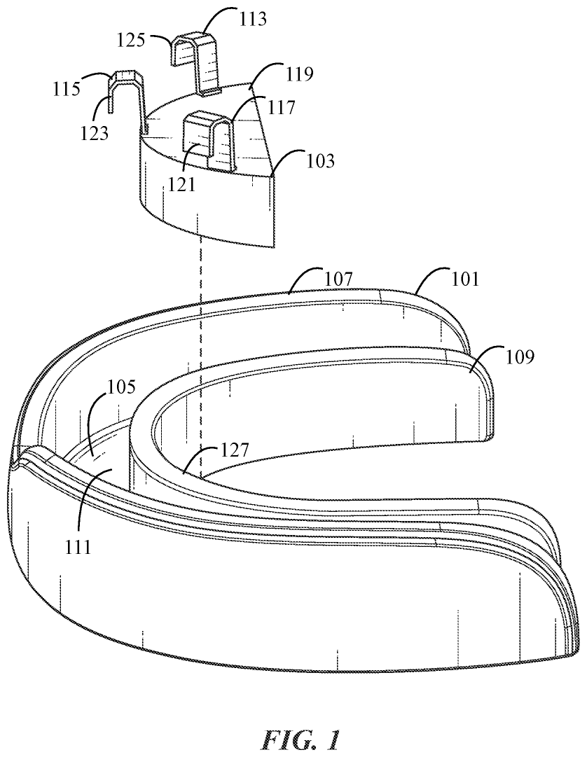

FIG. 1 illustrates an exploded view of the mouthguard 101 and inhaler respirator 103, wherein the inhaler respirator 103 is separated from the mouthguard 101. The mouthguard 101 may be configured in a similar manner as a traditional mouthguard having a bottom 105, an outer wall 107, and an inner wall 109 forming a channel 111 to accept the teeth of the user. The bottom 105, outer wall 107, and inner wall 109 form a protective barrier for the teeth as well as providing a cushioned surface to protect the mouth in general when worn. The inhaler respirator 103 is removably couple to the mouthguard 101 using clips 113,115,117. The inhaler respirator 103 may be shaped as a semi-circle or similar shape to conform to the shape of the inner wall 109. The 113,115,117 attached to the top surface 119 of the inhaler respirator 103 and extends about perpendicular therefrom. When coupling the inhaler respirator 103 to the mouthguard 101 the inhaler respirator 103 is slid down the inner wall 109 until the hook portions 121,123,125 of the clips 113,115,117 engaged with the top edge 127 of the inner wall 109.

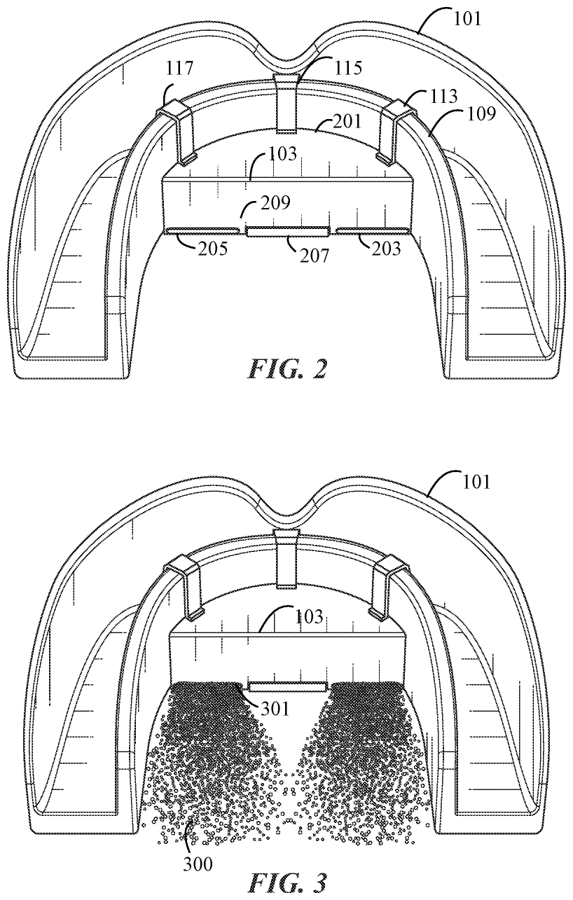

FIG. 2 illustrates a perspective view of the top of the mouthguard 101 and inhaler respirator 103, wherein the inhaler respirator 103 is coupled to the mouthguard 101. The clips 113,115,117 may form a friction fit with the inner wall 109 of the mouthguard 101 to retain the inhaler respiration in position at the front portion 201 of the mouthguard. Openings 203,205 are provided on the rear side 207 of the inhaler respirator 103 to expel the medication once the button 209 is pressed by the user. The button is positioned on the bottom of the inhaler respirator 103 such that it may be easily accessed and pressed by the user's thumb. The mouthguard 101 and inhaler respirator 103 are formed to have an ergonomic fit within the user's mouth.

In some embodiments, the ergonomic fit may be aided by user-specific molding of the mouthguard and/or inhaler respirator to the user's mouth.

FIG. 3 illustrates a perspective view of the mouthguard 101 and inhaler respirator 103 expelling a medication 300. The medication 300 is expelled from the openings 203,205 and into the user's mouth. The medication 300 may then be drawn into the lungs by the user inhaling. The medication may be expelled by a propellant 301, such as a CO2 cartridge positioned within the inhaler respirator 103. The inhaler respirator 103 have an interior cavity to retain the medication 300 and propellant 301 therein.

In some embodiments, the inhaler respirator 103 may include a rechargeable receptacle for to allow the user to reload the inhaler respirator 103 with additional medication 300.

In some embodiments, the inhaler respirator 103 may be disposable, such that when the medication 300 has been utilized and is depleted, it may be replaced with a new inhaler respirator 103 having a full dose or multiple doses of medication 300.

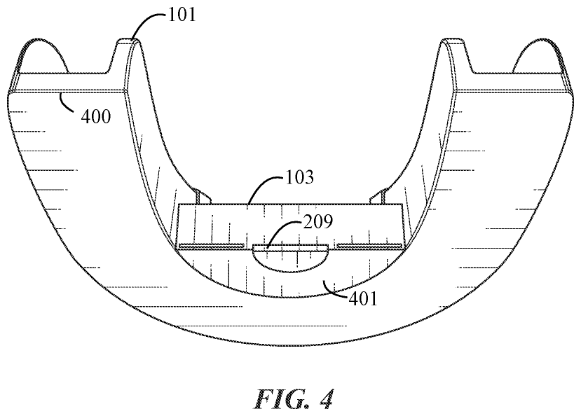

FIG. 4 illustrates a perspective view of the bottom 400 of the mouthguard 101 and inhaler respirator 103, wherein the inhaler respirator 103 is coupled to the mouthguard. The bottom surface 401 of the inhaler respirator includes a button 209 which can be pressed to release the medication. The button 209 may be constructed by various means such that the button may be a single-press button, multi-stage button (such as to release variable amount of medication) or may be pressed multiple times throughout use of the device.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety to the extent allowed by applicable law and regulations. The systems and methods described herein may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

The foregoing is provided for purposes of illustrating, explaining, and describing embodiments of this disclosure. Modifications and adaptations to these embodiments will be apparent to those skilled in the art and may be made without departing from the scope or spirit of this disclosure.

As used herein and in the appended claims, the singular forms “a”, “an”, and “the” include plural referents unless the context clearly dictates otherwise.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described herein. A variety of modifications and variations are possible in light of the above teachings without departing from the following claims.

What is claimed is:

1. A mouthguard including an inhaler respirator to deliver a medication, comprising:
a mouthguard;
an inhaler respirator including one or more clips to removably coupled to the mouthguard;
a propellant stored within the inhaler respirator to expel a medication.

2. The device of claim 1, wherein the medication is an asthma medication.

3. The device of claim 1, wherein the mouthguard is comprised of an inner wall, an outer wall, and a bottom to form a channel.

4. The device of claim 3, wherein the inner wall forms a friction fit with the one or more clips to retain the inhaler respirator in position.

5. The device of claim 1, wherein the inhaler respirator includes a button to be selectively pressed to expel the medication.

6. The device of claim 5, wherein the medication is expelled through one or more openings positioned on a rear side of the inhaler respirator.

7. The device of claim 1, wherein the one or more clips protrude perpendicular to the top surface of the inhaler respirator.

8. The device of claim 1, wherein the propellant is carbon dioxide.

9. A mouthguard including an inhaler respirator to deliver a medication, comprising:
a mouthguard to protect the mouth from impact, the mouthguard to releasably engage with the teeth and gums of a user;
an inhaler respirator including one or more clips to removably coupled to the mouthguard;
a propellant stored within the inhaler respirator to expel a medication through one or more openings.

10. The device of claim 9, wherein the medication is an asthma medication.

11. The device of claim 9, wherein the medication is a supplement.

12. The device of claim 9, wherein the mouthguard is comprised of an inner wall, an outer wall, and a bottom to form a channel, the channel to receive the teeth of the user.

13. The device of claim 12, wherein the inner wall forms a friction fit with the one or more clips to retain the inhaler respirator in position, wherein the one or more clips allow for the selective replacement of the inhaler respirator.

14. The device of claim 9, wherein the inhaler respirator includes a button to be selectively pressed to expel the medication.

15. The device of claim 9, wherein the one or more clips protrude perpendicular to the top surface of the inhaler respirator.

16. The device of claim 9, wherein the propellant is carbon dioxide.

17. The device of claim 9, wherein the inhaler respirator is capable of expelling multiple doses of the medication.

18. The device of claim 9, wherein the inhaler respirator is rechargeable to replenish the medication.
